(19) United States
(12) Patent Application Publication
(10) Pub. No.: US 2020/0339449 A1
(43) Pub. Date: Oct. 29, 2020

(54) TEST APPARATUS FOR A WASTE WATER TREATMENT SYSTEM

(71) Applicant: CDEnviro Limited, Cookstown, County Tyrone (GB)

(72) Inventor: Sean Dobbs, Cookstown, County Tyrone (GB)

(21) Appl. No.: 16/326,364

(22) PCT Filed: Aug. 7, 2017
(86) PCT No.: PCT/EP2017/069975

§ 371 Date: Feb. 18, 2019

(30) Foreign Application Priority Data
Aug. 18, 2016 (GB) 1614133.5

(51) Int. Cl. C02F 1/52; G01N 1/20; G01N 15/04; G01N 33/18

(52) U.S. Cl. CPC C02F 1/5209; G01N 1/2035; G01N 15/04; G01N 2001/1025; C02F 2209/11; C02F 2209/44; G01N 33/18

(57) ABSTRACT

A test apparatus for a waste water treatment system includes a fluid passageway having an inlet communicating with a first region of a flow path of the waste water treatment system and an outlet communicating with a second region of the flow path. At least a portion of the fluid passageway defines a test chamber within which a test may be carried out. The inlet and outlet are arranged within the flow path such that a pressure differential exists between the inlet and outlet such that waste water is caused to be diverted from the flow path through the test chamber from the inlet to the outlet of the fluid passageway. First and second valves may be provided in the fluid passageway upstream and downstream of the test chamber, the valves being operable to trap a test sample of waste water within the test chamber during a test process.

### CROSS REFERENCE TO RELATED APPLICATIONS

The present application is a § 371 national stage of International Application PCT/EP2017/069975, filed Aug. 7, 2017, which claims priority benefit of U.K. Pat. Application Ser. No. 1614133.5, filed Aug. 18, 2016, both of which are hereby incorporated herein by reference in their entireties.

### FIELD OF THE INVENTION

This invention relates to a test apparatus for a waste water treatment system and, in particular, to a test apparatus for testing the correct dosage of a flocculating agent added to waste water to be passed into a settling tank.

### BACKGROUND OF THE INVENTION

Waste water streams containing entrained solid impurities and contaminants in suspension are produced from numerous quarrying, mining, chemical or industrial processes. It is often desirable to reuse such water, particularly in regions prone to water shortages.

Before the waste water can be re-used, the solid impurities and contaminants (referred to as “fines”) must be removed from the water. This is typically done by passing the water into a settling tank wherein the fines are able to settle out under the action of gravity. A flocculating agent is typically added to the waste water to which the fines bind to bring them out of suspension.

The amount of flocculating agent added to the waste water needs to be accurately metered to ensure efficient separation of the fines from the waste water without wastage of the relatively expensive flocculating agent. It is therefore desirable to periodically test the settlement rate of the fines in the waste water to determine that the correct amount of flocculating agent is present.

This testing is typically achieved by withdrawing waste water from the settlement tank into a vertically arranged transparent section of pipework defining a test chamber, trapping a sample of the waste water in the test chamber and timing how long it takes for the solid material within the sample in the test chamber to fall out of suspension, typically by using a photo-detector mounted at a predetermined height on the test chamber.

In a known arrangement waste water is withdrawn under gravity from the settlement tank to flow through the test chamber before passing into a collection sump. When it is desired to carry out a test, valves upstream and downstream of the test chamber are closed and a timer is started to determine the time interval before the photo-detector detects clear water at the predetermined height in the test chamber. Typically once a test has been completed, the downstream valve is opened (keeping the upstream valve closed) and a further valve is opened to pass clean water through the test chamber to flush the test chamber. The valves are then shut to close off the test chamber and waste water collected in the collection sump is pumped back up into the settlement tank.

A problem with such known systems is that the height difference between the test chamber and the settlement tank can be significant, resulting in a high flow rate through the test chamber when both the upstream and downstream valves are open. When the upstream and downstream valve are then suddenly closed the sudden change in flow velocity within the test chamber can create turbulent conditions, breaking up flocculated fines in the test sample and resulting in a slower than representative settlement rate.

The additional pipework, pumps and sump also takes up space and adds to the cost of the system.

### SUMMARY OF THE INVENTION

According to a first aspect of the present invention there is provided a test apparatus for a waste water treatment system comprising a fluid passageway having an inlet communicating with a first region of a flow path of the waste water treatment system and an outlet communicating with a second region of the flow path, downstream of the first region, at least a portion of the fluid passageway defining a test chamber within which a test may be carried out, wherein the inlet and outlet are arranged within the flow path such that a pressure differential exists between the inlet and outlet whereby waste water is caused to be diverted from the flow path through the test chamber from the inlet to the outlet of the fluid passageway.

First and second valves may be provided in the fluid passageway upstream and downstream of the test chamber, the valves being operable to trap a test sample of waste water within the test chamber during a test process. In one embodiment the test chamber may be arranged substantially vertically, at least one sensor being associated with the test chamber for sensing the turbidity of the waste water within the test chamber at a predetermined level therein, a controller incorporating a timer being provided for determining the time taken for settlement of fines out of suspension in the waste water trapped within the test chamber when the first and second valves are closed as determined by the detection of an absence of reduced level of turbidity at the predetermined level by the at least one sensor as a measure of the amount of a flocculating agent in the waste water sample. In an exemplary embodiment a first sensor may be provided at a first height, adjacent the top of the test chamber, and a second sensor is provided at a second height, below the first height and adjacent a lower end of the test chamber, the timer being started when the first sensor detects clear water and stopped when the second sensor detects clear water. Each sensor may comprise a photo-detector detecting the transmission of light through the waste water within the test chamber. The inlet of the fluid passageway may be located below the outlet such that waste water flows upwardly through the test chamber from the inlet to the outlet. The flow path may comprise an upwardly extending pipe.

Optionally, the inlet of the fluid passageway communicates with the flow path at an outer side of a bend in the flow path. The inlet may extend tangentially from the bend in the flow path such that waste water enters the inlet due to the inertia of the waste water.

It is also contemplated that the shape of the outlet of the fluid passageway in the flow path may be adapted to create a low pressure region in the vicinity of the outlet. In one embodiment the outlet of the fluid passageway in the flow path may include an outlet opening in the flow path and a wall extending into the flow path on an upstream side of the outlet opening, the wall generating a low pressure region adjacent the outlet opening. The wall may comprise a semi-circular wall extending around an upstream side of the outlet opening around which wall the waste water in the flow path is constrained to flow.

At least a portion of the test chamber may be formed from a transparent material.

A clean water inlet may be provided for supplying clean water to an upstream end of the test chamber under the control of a further valve for flushing the test chamber following the test.

According to a further aspect of the present invention there is provided a waste water treatment system incorporating a test apparatus in accordance with the first aspect of the invention.

In one embodiment the apparatus may include a mixing chamber within which a flocculating agent is mixed with a waste water stream and a settlement tank downstream of the mixing chamber wherein fines settle out from the waste water, the flow path comprises a pipeline extending from the mixing chamber to the settlement tank.

The flow path is envisioned to extend in an upwardly direction from the mixing chamber to the settlement tank. The test chamber may extend substantially parallel to the flow path between the inlet and the outlet of the fluid passageway.

The pipeline may include a bend, the inlet of the fluid passageway being located on an outer side of the bend. The inlet may extend substantially tangentially from the bend.

The outlet may be located in a side wall of the pipeline at a location downstream of the inlet, a wall being provided on an upstream side of the outlet extending into the pipeline, the wall generating a low pressure region in the pipeline adjacent the outlet.

These and other objects, advantages and features of the invention will become apparent upon review of the following specification in conjunction with the drawings.

### BRIEF DESCRIPTION OF THE DRAWINGS

A waste water treatment system incorporating a test apparatus in accordance with an embodiment of the present invention will now be described, by way of example only, with reference to the accompanying drawings, in which:—

FIG. 1 is a perspective view of a waste water treatment system incorporating a test apparatus in accordance with an embodiment of the present invention; and

FIG. 2 is a sectional view through a part of the system of FIG. 1 showing the test apparatus in more detail.

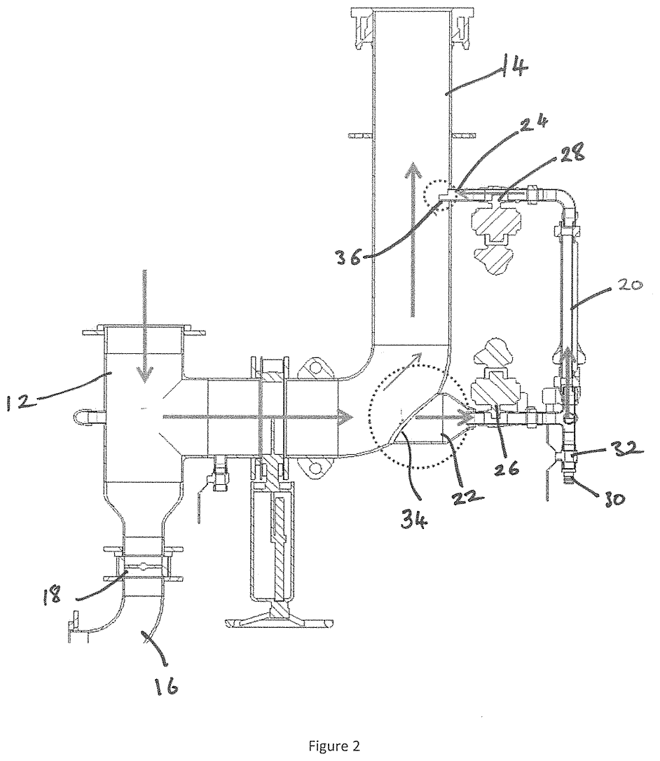

### DESCRIPTION OF THE PREFERRED EMBODIMENTS

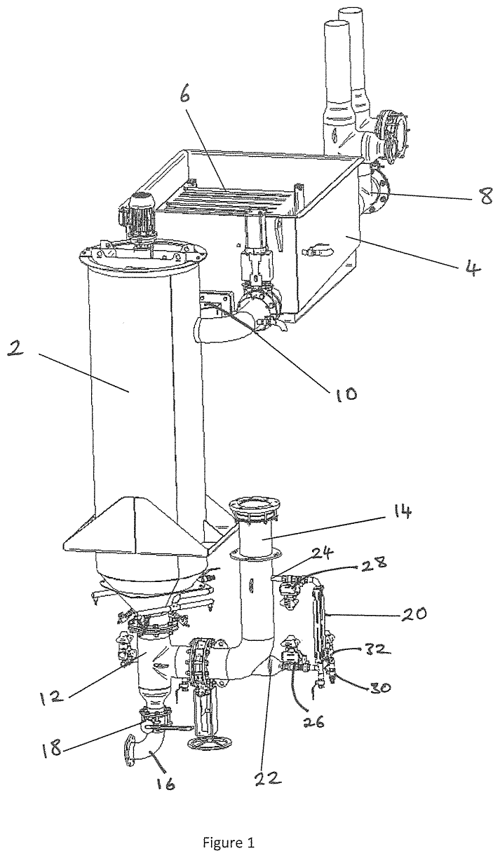

FIG. 1 illustrates a water treatment system incorporating a test apparatus in accordance with an embodiment of the present invention. The waste water treatment system shown in FIG. 1 comprises a dosing and mixing apparatus 2 within which a flocculating agent and/or other chemical is added to and mixed with a waste water stream upstream of a settling tank.

A de-aerating apparatus 4 is provided upstream of the mixing apparatus 2 to remove entrained air from the waste water. There is typically a lot of air entrained in waste water discharged from sand and aggregate washing and grading processes and other washing processes and this entrained air inhibits the fast and effective settlement of flocculated solids. By removing air from the waste water prior to addition of the flocculating agent, the effectiveness of the flocculating agent is enhanced, potentially allowing the use of a smaller settlement tank.

The de-aerating chamber 4 incorporates a dividing wall 6 dividing the chamber 4 into first and second regions, an the inlet 8 communicating with the first region of the chamber 4 and an outlet 10 communicating with the second region of the chamber 4, the dividing wall 6 defining a weir over which waste water has to pass to travel from the inlet 8 to the outlet 10 of the chamber 4. This has the effect of reducing turbulence in the waste water.

The waste water, to which a predetermined dose of a flocculating agent has been added, passes out of a lower end 12 of the mixing apparatus 2 and passes up a vertically arranged delivery pipe 14 to be delivered into a settlement tank (not shown).

A drain 16 is provided below the lower end 12 of the mixing apparatus 2, drain 16 being normally closed by a shut off valve 18.

A test chamber 20, defined by a vertically arranged transparent pipe, is arranged in parallel to the delivery pipe 14, having an inlet 22 at a lower end of the delivery pipe 14 and an outlet 24 adjacent an upper end of the delivery pipe 14, outlet 24 being arranged perpendicular to the delivery pipe 14. The test chamber 20 may be use to test the rate of settlement of the fines out of suspension as a test of the dosage of flocculating agent within a sample. At least one light source may be provided for directing light through the test chamber and a respective photo-detector may be provided for determining the turbidity of the sample contained therein by receiving light from the respective light source once it has passed through the waste water in the test chamber 20. The light source and photo-detector may be provided at a predetermined height on the test chamber 20, whereby the water at the height becomes clear when settlement of the fines within the sample is complete. The light source and detector may operate in the infra-red range. In the illustrated embodiment a first detector is provided at a first height, such as adjacent the top of the test chamber 20, and a second detector is provided at a second height, below the first height and optionally adjacent a lower end of the test chamber 20.

A first valve 26 is provided for controlling communication between the inlet 22 and the test chamber 20 and a second valve 28 is provided for controlling communication between the test chamber 20 and the outlet 24. A further valve 32 is provided for controlling communication between a fresh water supply 30 and the lower end of the test chamber 20 for flushing the test chamber 20, as will be described in more detail below.

The inlet 22 leading from the delivery pipe 14 into the test chamber 20 is arranged in a high pressure region of the delivery pipe 14, more specifically on the outside of a right angle bend 34 at the lower end of the delivery pipe, the inlet 22 extending tangentially from an outer side of the bend 34 where the velocity and pressure of the waste water is at a maximum.

The shape of the outlet 24 is such that a low pressure region is generated within the delivery pipe 14 in the vicinity of the outlet 24. This is achieved by providing a curved wall 36 extending into the delivery pipe 14 on an upstream side of the outlet 24 relative to the normal direction of flow of waste water within the delivery pipe 14, wall 36 shielding the outlet 24 from the flow within the delivery pipe 14 and creating a low pressure region at the outlet 24 as the water flows around the wall 36.

The pressure differential between the inlet 22 and outlet 24 causes water to flow upwardly through the test chamber 20 when the first and second valves 26,28 are both open as waste water flows upwardly through the delivery pipe 14.

During normal operation of the waste water treatment system the first and second valves 26,28 and the further valve 32 remain closed and waste water passes from the mixing apparatus 2, through the outlet 12 and up the delivery pipe 14 to be delivered into the settlement tank.

Periodically, under the control of a PLC, the valve 26,28 are opened to initiate a test cycle. Once the first and second valves 26 and 28 are opened, a portion of the waste water flows through into the inlet 22, through the test chamber 20 and returns into the delivery pipe 14 due to the pressure differential between the inlet 22 and outlet 24, as discussed above. After a predetermined period of time the first and second valves 26,28 are then closed to trap a sample of waste water and entrained fines within the test chamber 20.

The fines begin to settle out of the waste water within the test chamber 20 and collect in the bottom of the test chamber 20. A timer may be started when the first detector detects clear water and stopped when the second detector detects clear water. The period of time before the second photo-detector detects clear water is indicative of the correct dosage of flocculating agent.

Once the test cycle has completed the first valve 26 remains closed while the second valve 28 is opened and the further valve 32 is opened to supply fresh water into the lower end of the test chamber 20 to flush the test chamber 20, the settled fines and the flushing water passing into the delivery pipe 14 via the outlet 24. Once the flushing process has completed after a predetermined period of time the further and second valves 28,32 are closed, once again isolating the test chamber 20 from the delivery pipe 14 until the next test cycle is initiated.

The test apparatus according to the present invention requires no sump or pump or associated level sensors or pipework and is considerably more compact than prior art systems. The test chamber 20 is never empty (either containing waste water from the delivery pipe 14 or clear water from the fresh water supply 30. Therefore the liquid within the test chamber acts as a damper when the first and second valves 26,28, are first opened, avoiding turbulence and resulting in minimal to no flocculated solids break up. The fact that the flow of waste water through the test chamber 20 occurs in parallel to the delivery pipe 14, waste water being sampled from and returned to the deliver pipe 14, provides more gentle and slower flow rates through the test apparatus, maintaining floc structure and avoiding sudden pressure changes and associated turbulence, allowing faster test cycle times compared to prior art systems, where delays have to be built in to allow turbulence to settle.

The invention is not limited to the embodiment(s) described herein but can be amended or modified without departing from the scope of the present invention, which is intended to be limited only by the scope of the appended claims as interpreted according to the principles of patent law including the doctrine of equivalents.

1. A test apparatus for a waste water treatment system, the test apparatus comprising:
a fluid passageway having an inlet communicating with a first region of a flow path of the waste water treatment system and an outlet communicating with a second region of the flow path, downstream of the first region, at least a portion of said fluid passageway defining a test chamber within which a test may be carried out, wherein said inlet and said outlet are arranged within the flow path such that a pressure differential exists between said inlet and said outlet whereby waste water is caused to be diverted from the flow path through the test chamber from the inlet to the outlet of said fluid passageway.

2. The test apparatus of claim 1, wherein first and second valves are provided in the fluid passageway upstream and downstream of said test chamber, said valves being operable to trap a test sample of the waste water within said test chamber during a test process.

3. The test apparatus of claim 2, wherein said test chamber is arranged substantially vertically and comprises:
at least one sensor associated with said test chamber for sensing turbidity of the waste water within said test chamber at a predetermined level therein; and
a controller incorporating a timer provided for determining time taken for settlement of fines out of suspension in the waste water trapped within said test chamber when said first and second valves are closed as determined by detection of an absence of or reduced level of the turbidity at the predetermined level by said at least one sensor as a measure of an amount of a flocculating agent in the test sample of the waste water.

4. The test apparatus of claim 3, wherein a first sensor is provided at a first height adjacent the top of the test chamber, and a second sensor is provided at a second height, below said first height and adjacent a lower end of the test chamber, said timer being started when said first sensor detects clear water and stopped when said second sensor detects clear water.

5. The test apparatus of claim 3, wherein said at least one sensor comprises a photo-detector detecting transmission of light through the waste water within said test chamber.

6. The test apparatus of claim 1, wherein said inlet of the fluid passageway is located below said outlet such that the waste water flows upwardly through said test chamber from said inlet to said outlet.

7. The test apparatus of claim 6, wherein the flow path comprises an upwardly extending pipe.

8. The test apparatus of claim 1, wherein said inlet of the fluid passageway communicates with the flow path at an outer side of a bend in the flow path.

9. The test apparatus of claim 8, wherein said inlet extends tangentially from the bend in the flow path such that the waste water enters said inlet due to inertia of the waste water.

10. The test apparatus of claim 1, wherein the shape of said outlet of said fluid passageway in the flow path is adapted to create a low pressure region in a vicinity of said outlet.

11. The test apparatus of claim 10, wherein said outlet of said fluid passageway in the flow path includes an outlet opening in the flow path and a wall extending into the flow path on an upstream side of said outlet opening, said wall generating a low pressure region adjacent said outlet opening.

12. The test apparatus of claim 10, wherein said wall comprises a semi-circular wall extending around the upstream side of said outlet opening, and wherein the waste water in the flow path is constrained to flow around said wall.

13. The test apparatus of claim 1, wherein at least a portion of said test chamber is formed from a transparent material.

14. The test apparatus of claim 1, further comprising a water inlet for supplying clean water to an upstream end of said test chamber under control of a valve for flushing said test chamber following the test.

15. A waste water treatment system incorporating a test apparatus comprising a fluid passageway having an inlet communicating with a first region of a flow path of said waste water treatment system and an outlet communicating with a second region of said flow path, downstream of said first region, at least a portion of said fluid passageway defining a test chamber within which a test may be carried out, wherein said inlet and said outlet are arranged within said flow path such that a pressure differential exists between said inlet and said outlet whereby waste water is caused to be diverted from said flow path through said test chamber from said inlet to said outlet of the fluid passageway.

16. The system of claim 15, wherein said apparatus comprises a mixing chamber within which a flocculating agent is mixed with a waste water stream and a settlement tank downstream of said mixing chamber, wherein fines settle out from the waste water, said flow path comprises a pipeline extending from said mixing chamber to said settlement tank.

17. The system of claim 16, wherein said flow path extends in an upwardly direction from said mixing chamber to said settlement tank.

18. The system of claim 16, wherein said test chamber extends substantially parallel to the flow path between said inlet and said outlet of the fluid passageway.

19. The system of claim 16, wherein said pipeline includes a bend, said inlet of the fluid passageway being located on an outer side of said bend.

20. The system of claim 19, wherein said inlet extends substantially tangentially from said bend.

21. The system of claim 16, wherein said outlet is located in a side wall of said pipeline at location downstream of said inlet, wherein a wall is provided on an upstream side of said outlet extending into said pipeline, and wherein said wall generates a low pressure region in said pipeline adjacent said outlet.
